(19) United States
(12) Patent Application Publication
(10) Pub. No.: US 2007/0167793 A1
(43) Pub. Date: Jul. 19, 2007

(54) Method and system for enhancing spectral doppler presentation

(75) Inventors: Praveen Dala-Krishna, Sicklerville, NJ (US); David A. Jenkins, Flanders, NJ (US); Charles Bryan Byrd, Medford, NJ (US)

Correspondence Address: HANSEN HUANG TECHNOLOGY LAW GROUP, LLP, 1725 EYE STREET, NW, SUITE 300, WASHINGTON, DC, 20006, US

(73) Assignee: EP MedSystems, Inc., West Berlin, NJ (US)

(21) Appl. No.: 11/302,391

(22) Filed: Dec. 14, 2005

(51) Int. Cl. A61B 8/00

(52) U.S. Cl. 600/455

(57) ABSTRACT

A method and system for enhancing Doppler spectrum displays is described. Two or more colors are utilized to represent the power scale in the spectral Doppler display. Doppler signals from stronger reflectors of ultrasound, such as cardiac muscle or vessel walls, can then be assigned a different color than Doppler signals from weaker reflectors, such as blood, allowing for better understanding and interpretation of the Doppler spectra.

### BACKGROUND

1. Field

The present invention relates generally to medical imaging systems, and more particularly to a method and apparatus for enhancing the presentation of Doppler signals.

2. Background

Ultrasound devices have been developed and refined for the diagnosis and treatment of various medical conditions. Such devices have been developed, for example, to track the magnitude and direction of motion of moving objects, and/or the position of moving objects over time. By way of example, Doppler echocardiography is one ultrasound technique used to determine motion information from the recording and measurement of Doppler data for the diagnosis and treatment of cardiac conditions, and is described in greater detail below.

The Doppler principle, as used in Doppler echocardiography, generally involves exploiting an observed phenomenon that the frequency of reflected ultrasound pulses is altered by a moving object, such as moving tissue or blood cells. This alteration, or change, in frequency is generally referred to as a Doppler shift. The magnitude of the frequency change, or Doppler shift, is related to the velocity of the moving object from which the ultrasound pulses are reflected. The polarity of the frequency change, or Doppler shift, is related to the direction of motion relative to the ultrasound source: a positive frequency shift (increase) indicates the motion is towards the ultrasound sensor and a negative frequency shift (decrease) indicates that the motion is away from the ultrasound sensor. That is, if the object is moving towards the source of the Doppler signal, the reflected ultrasound pulses are compressed, resulting in an increase in frequency of the pulses. Likewise, if the object is moving away, the reflected ultrasound pulses are expanded, resulting in a decrease in frequency of the pulses. As such, the magnitude and polarity of the Doppler shift can be used to track the magnitude and direction of motion of moving objects.

Treatment and diagnosis techniques operating on the Doppler principle generally involve one of two types of Doppler signals, either continuous wave (CW) Doppler, or pulsed wave (PW) Doppler.

In general, CW Doppler techniques involve continuously transmitting an ultrasound signal and continuously receiving the reflections, or echoes, of the transmitted signals that are reflected, or backscattered, from objects that are in a region where the transmitted beam overlaps with a region where signals can be received by a receiver. Because the Doppler signals are continuously transmitted and received, it is not possible to differentiate, or separate, Doppler signals from objects that are at different locations within the overlap region that is common to both the transmitter and receiver. In contrast, PW Doppler techniques involve transmitting sets of ultrasound pulses and turning on a receiver to detect the reflections of the transmitted pulse for only a portion of the time between sets of pulses. This technique, also referred to as “gating”, turns the receiver “on” following a delay after the pulse is transmitted, where the length of the delay between the transmission and gating the receiver on corresponds to a first round trip distance along the ultrasound beam to the area of interest. Thus, by “gating” the receiver, turning the receiver on and off at desired times relative to a transmission, only signals from a “range” within the overlap region that is common to both the transmitter and receiver are received. The gate times correspond to the time it takes for the ultrasound signal to travel to and the reflected signal to travel back to the receiver from the desired range within the common region. This technique is also referred to as “range gating” or “time gating.”

The selection of CW Doppler or PW Doppler for a particular application depends on the requirements of the application at hand, as each technique has features and limitations readily apparent to those of skill in the art.

A technique that has been used to improve PW Doppler is the use of color in presenting the Doppler information. For example, in a PW Doppler based scan regions of interest can be superimposed with a color scale based on velocity, or direction of motion. As such, color Doppler can be thought of as an enhanced PW Doppler scan. The aforementioned Doppler techniques have been applied to the diagnosis and treatment of cardiac conditions, and can be grouped together and referred to generally as echocardiography.

Ultrasound imaging techniques include a class generally referred to as brightness mode (“B-Mode”) displays. In general, to generate a B-Mode display, the time interval between the transmission of a PW ultrasound pulse and the return of its echo is measured and used to determine the distance of a given object from the ultrasound transducer. The signal intensity is also measured. A display is then rendered from a collection of the ultrasound data, where the position of each “dot” corresponds to the distance from the ultrasound transducer of a given object, and the brightness of each “dot” corresponds to the signal strength at that position.

Another class of ultrasound imaging techniques is generally referred to as motion mode (“M-Mode”) displays. To generate an M-Mode display, the time interval between a first ultrasound pulse and the return of its echo, corresponding to depth, is plotted along one axis. Subsequent time intervals for subsequent ultrasound pulses (and their corresponding echoes) are then plotted along another axis, corresponding to time. This type of plot graphically depicts movement of a given object over time. Such a technique is described in U.S. Pat. No. RE37,088, which is incorporated by reference herein in its entirety.

The aforementioned ultrasound imaging techniques have given clinicians a wide variety of tools with which to diagnose and treat various medical conditions, such as the noted cardiac conditions. These tools are limited, however, in their ability to discern between various structures, and their ability to accurately track (and display) a moving structure amongst a plurality of moving structures.

Thus, a need exists for enhanced methods and apparatus for processing ultrasound signals and images. Other problems with the prior art not described above can also be overcome using the teachings of the present invention, as would be readily apparent to one of ordinary skill in the art after reading this disclosure.

### SUMMARY

Embodiments disclosed herein address the above stated needs by providing methods and apparatus for enhancing the processing of ultrasound signals and images. The techniques include a method and apparatus for processing Doppler signals which includes determining the signal strength of received Doppler signals. Then assigning a color to the received Doppler signals in accordance with the strength of the received Doppler signal. In one aspect, signals from stronger reflectors are represented in different colors than those from weaker reflectors.

In one embodiment, techniques for generating Doppler spectral displays are described, wherein Doppler signals from any strong reflectors, such as tissue, within a sample volume are presented, or displayed, in different colors than signals from weaker reflectors, such as blood. An aspect is that differentiation between different types of reflector material may be obtained by displaying frequency components with different amplitudes, or power levels, in different colors.

Another embodiment includes an ultrasound scanner, with Doppler capabilities, which has the capability to represent Doppler signals in a color scale through a functionality similar to a “look up table.” The ultrasound scanner can include various techniques of transmitting and receiving ultrasonic signals from the structure in question. For example, the ultrasound scanner may include a plurality of transducer configurations, such as single crystal transducers, single-dimensional array transducers, and multi-dimensional array transducers. The ultrasound transducer may also be included in a catheter. In addition, different power levels in the processed signal may be represented in two or more colors, and a color scale can be dynamically generated or the color scale may have been previously set up in the system through any combination of hardware or software.

Yet another embodiment provides an ultrasound scanner wherein a user can choose, or map, colors to be used to represent the power scale. A further embodiment can allow the user to choose a discrete or continuous range of linear or non-linear mapping techniques, wherein the various power levels of the received and processed signals are mapped in multiple linear or nonlinear ways to a user selected or static color scale for representation.

Other embodiments can include additional workstation(s), or computer(s), employed in conjunction with a ultrasonic scanning mechanism. The processing of the Doppler signals can occur either in the ultrasonic scanner, or the additional workstation(s) or computer(s), or both. In addition, the processed Doppler signal. or spectrum, can be displayed on the ultrasonic scanner display or the additional workstation(s) or computer(s) display, or both.

Yet other embodiments can include an offline workstation, or computer, that may receive data from an ultrasonic interrogation device and process Doppler data and carry out the described mapping on the data in a non-real-time situation.

Other features and advantages of the present invention should be apparent from the following description of exemplary embodiments, which illustrate, by way of example, aspects of the invention.

### BRIEF DESCRIPTION OF THE DRAWINGS

FIG. 1 is an illustration of an ultrasound sector.

FIG. 2 is a graph illustrating the Doppler signals that would be received from individual components of the heart valve and blood.

FIG. 3 is an illustration of a composite Doppler signal of FIG. 2.

FIG. 4 is a graphical representation of a linear mapping different power levels to different colors and different brightness levels.

FIG. 5 is an illustration of the composite Doppler signal of FIG. 3 applying the color mapping of FIG. 4.

FIG. 6 is graphical representation of an example of non-linear mapping of received signal strength to color.

FIG. 7 is an illustration of the composite Doppler signal applying the color mapping of FIG. 6.

FIG. 8 is graphical representation of another example of non-linear mapping of received signal strength to a multi-colored scale.

FIG. 9 is an illustration of the composite Doppler signal applying the color mapping of FIG. 8.

FIG. 10 is a graphical representation of yet another example of non-linear mapping of received signal strength to a multi-colored scale.

FIG. 11 is an illustration of the composite Doppler signal applying the color mapping of FIG. 10.

FIG. 12 is a block diagram illustrating an embodiment of a Doppler scanner system constructed in accordance with the present invention.

FIG. 13 is a block diagram of the Doppler scanner system of FIG. 12 and includes a workstation.

FIG. 14 is a block diagram of another embodiment of a Doppler scanner system.

FIG. 15 is a block diagram of yet another embodiment of a Doppler scanner system.

FIG. 16 is a block diagram illustrating another embodiment of a Doppler scanner system constructed in accordance with the present invention.

FIG. 17 is a flow diagram illustrating a method of enhancing the presentation of Doppler signals.

FIG. 18 is a block diagram illustrating a method of processing Doppler signals.

### DETAILED DESCRIPTION

The word “exemplary” is used herein to mean “serving as an example, instance, or illustration.” Any embodiment described herein as “exemplary” is not necessarily to be construed as preferred or advantageous over other embodiments.

Techniques for enhancing the representation of Doppler signals are described. The techniques include improving the representation of Doppler signals received from different types of material, for example, tissue and blood.

Conventionally, in a PW Doppler system, an ultrasound pulse is transmitted in a particular direction. A receiver is then turned “on” and “off” so that only a portion of the volume that the ultrasound pulse is transmitted through is sampled. This technique of only receiving signals from a so-called sample volume, is also referred to as range gating. Range gating techniques are also used in ultrasound systems that use scanning probes and transducer. The sample volume represents a continuous range of distances from the surface of the ultrasound transducer along the ultrasound beam. Shifts in frequency of a reflection of the ultrasonic beam from the frequency of the transmitted beam are referred to as the Doppler shift. These frequency shifts occur due to the motion of any reflector, also termed as scatterers or backscatterers, and in a PW Doppler system are determined within the sample volume. Various ways of determining the Doppler shift are known to those of skill in the art.

These Doppler shifts in the reflected, or backscattered, ultrasound beam are directly correlated to the velocity of the backscatterers, with respect to the ultrasound transducer. Typically, the Doppler shift information is displayed such that the magnitude of the Doppler shift is plotted along a vertical (y) axis of a display, with respect to time indicated along a horizontal (x) axis. Thus, such a display, in effect, displays the range of frequency shifts from the fundamental transmitted frequency, and in turn the various velocities of motion of one or more reflectors through the sample volume are displayed. Further, the strength of the received signal at a particular (shifted) frequency may be used to control the brightness of a corresponding point of the display. For example, the strength at a particular (shifted) frequency may be mapped to either a linear or a non-linear gray scale level, thus defining the brightness or gray level of each point in the spectrum corresponding to the received signal strength at any given time.

For example, when blood passes through a vessel, the walls of the vessel expand and contract with every beat of the heart, known as the pulse. If a vessel wall is part of the sample volume, it results in a strong low frequency component in the signal, given that the blood in the vessel being interrogated flows at a higher velocity than the velocity of movement of the vessel wall. High pass filters, termed “wall-filters”, whose cut-off and pass bands can be actively adjusted either automatically, or by the user, have been extensively used to remove such unwanted signals. It should be noted that in such applications, the velocity of movement of the vessel wall is much less than the velocity of the flow within.

In conventional use of such a system, the system images a large field of tissue with relatively smaller fields of flow, such as flow through the arteries. Hence the sample volumes are relatively small compared to the total field of view. Given the anatomical stability of the relative position of the center of a blood vessel with respect to the transducer, the sample volume could be usually well positioned within the flow of interest while any incursions into the area of interest by the vessel wall could be filtered out by the wall filters.

A drawback with the above described technique occurs when considering intracardiac imaging. In intracardiac ultrasonic imaging, a significantly larger area of the image is taken over by flowing blood, thus necessitating larger sample volumes in many instances. In addition to the blood in the heart, the whole tissue/area under investigation is constantly in motion. In many cases, such as with the various valves of the heart, the tissue moves at considerable velocities. There can be instances, such as at the beginning of a ventricular contraction, when the velocity of the leaflets of the heart valve are comparable to the velocity of blood through the valve.

Procedures and techniques are described to provide better discrimination and presentation of Doppler signal information received from various types of materials. For example, techniques are described for improved discrimination and presentation of Doppler signal information when the Doppler signal includes information from relatively slow velocity tissue movement as well as information from higher velocity blood flow surrounding the tissue. In addition, techniques are described for improved discrimination and presentation of Doppler signal information when the Doppler signal includes information from tissue movement as well as information from blood that is moving at relatively the same velocity as the tissue. The techniques described can be used with various types of ultrasound systems. For examples, the techniques can be used with ultrasonic transducers that are used external to a person as well as catheter systems wherein the ultrasonic transducer enters a persons body.

It is well known to those of skill in the art that different biological materials interact differently with ultrasound. See P. N. T. Wells, Biomedical Ultrasonics pages 110-144 (Academic Press 1977). For example, soft tissues generally reflect ultrasound more strongly than blood. Thus, a signal received from moving tissue structures would typically be of a larger amplitude than a signal received from blood moving within, or around, the moving tissue structures. Using conventional processing, such situations result in strong Doppler signals from the moving tissue that may be indistinguishable from Doppler signals from blood, or in some cases, even be superimposed and thereby obscure the signals from the blood.

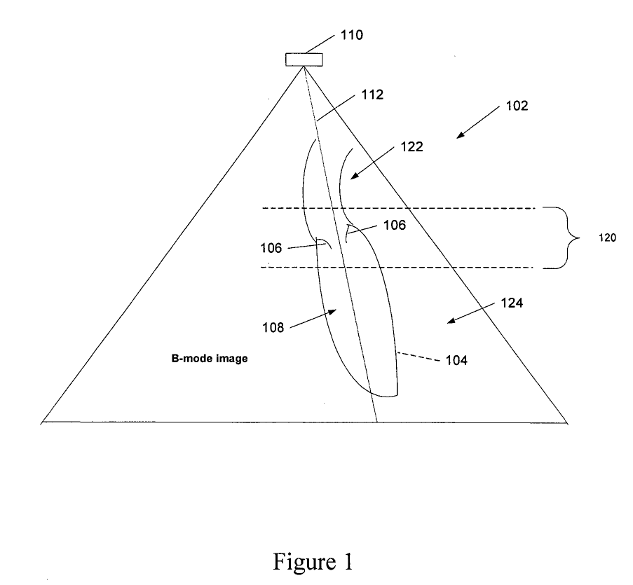

FIG. 1 is an illustration of an ultrasound sector 102. In this example, the ultrasound is being used to examine portions of a heart valve 104. The heart valve 104 includes leaflets 106 with blood 108 flowing through the valve 104 and valve leaflets 106.

As shown in FIG. 1, an ultrasound transceiver 110, that includes an ultrasound transmitter and an ultrasound receiver, transmits an ultrasound beam 112, or a plurality of beams. For example a narrow beam may be swept through an arc forming the sector, or multiple beams may be transmitted simultaneously to form the sector, or other techniques as is known to those of skill in the art. The ultrasound transducer may also be included in a steerable catheter where it is fitted on to the tip of the catheter and is used to view the interior anatomy of a heart to perform intra-cardiac ultrasonic imaging, a technique that has significantly improved the definition and clarity of views of diseased valves. For purposes of illustration a single instance of an ultrasound beam 112 is illustrated within the sector 102. As the ultrasound beam 112 propagates through the sector it interacts with the material within its path that includes the heart valve 104, heart valve leaflets 106, and blood 108.

In this example the ultrasound is range gated so that the region of interest 120 around the heart value leaflets 106, and the surrounding blood 108, are examined. Thus, the region of the sector 102 that the ultrasound beam 112 passes through before and after the area of interest, 122 and 124 respectively, are not examined and not displayed in the Doppler presentation.

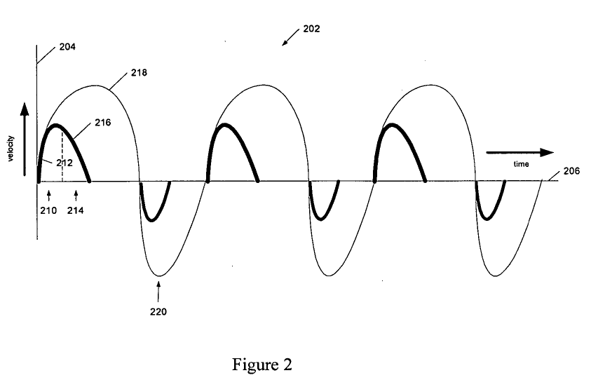

FIG. 2 is a graph illustrating the Doppler signals 202 that would be received from the individual components of the heart valve 104 and blood 108. As shown in FIG. 2, the graph has a vertical axis 204 that represents velocity as measured by the Doppler shift of the ultrasound beam 112 as it passes through the area of interest 120 that includes the heart value leaflets 106 and surrounding blood 108. The horizontal axis 206 represents time. In FIG. 2 the Doppler display 202 includes Doppler information from both the heart tissue, primarily the heart leaflets 106 and the blood 108 flowing through the leaflet.

To assist in explaining the velocities of the various materials of interest FIG. 2 shows two separate signals, one representing a Doppler signal that would be received from the valve leaflets 216 alone, and a second representing a Doppler signal that would be received from the blood flow through the valve 218 alone. The depiction of FIG. 2 is for illustration only because the two signals shown are actually received as a single combination, or composite, signal. That is, as illustrated, during the period 210 of the heart valve operation when the valve is opening and blood is starting to flow, the velocity of the valve leaflets is approximately the same as the velocity of the blood and the display of the two signals appears as a single curve 212. During the period 214 when the value leaflets begin to slow, until the value leaflets are fully opened, the valve leaflets are moving slower than the blood flowing through the valve, illustrated by curves 216 and 218 respectively. Because the velocity of the blood 218 is larger than the velocity of the valve leaflets 216, there is a separation between the two curves. During the region where the value leaflets close 220, there may be some regurgitation, or “backflow” through the value. In the example of FIG. 2, the backflow of the blood during this period is greater than the velocity of the valve leaflets, and therefore the two curves again separate with the blood flow curve 218 larger in amplitude than the valve leaflet curve 218. The Doppler signal then repeats itself.

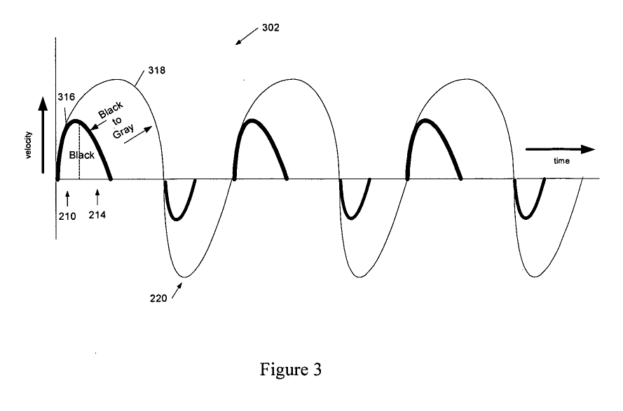

As noted, the discrete signal display in FIG. 2 is not possible in a conventional Doppler display because the Doppler “signal” is a combination of signals received from the heart valve leaflets and the blood, not two separate signals. FIG. 3 is an illustration of a composite Doppler signal such as shown in FIG. 2. In FIG. 3 the display of the Doppler signal is indicated with a gray scale to represent received signal strength, with a larger signal strength being indicated by a darker display. In the period 210 where the heart valve leaflets are moving at approximately the same velocity as the blood, the display of the heart valve leaflet signal 316 completely masks, or makes it very difficult to discriminate, the blood flow Doppler signal 318. In the period 214 when the value leaflets begin to slow, until the value leaflets are fully opened, the velocity of the blood 318 is larger than the velocity of the valve leaflets 316 and there is a separation between the two curves. But, because the amplitude of the signal received from the valve leaflets is typically much larger than the return from the blood, the display of the velocity of the valve leaflets 316 may mask, or interfere with the ability to discriminate the differences between the two flows. For example, as shown in FIG. 3 it is difficult to detect the weaker signal from the blood 318 during this period 214. It is also very difficult to discriminate between the velocity of the valve and the blood because there is a gradual change from black to dark gray to gray.

During the region where the valve leaflets close 220, if there is any regurgitation, or “backflow” through the valve, it may be masked and difficult to be detected. For example, as shown in FIG. 2, during the period when the valves closes 220, depending on the velocity of the backflow of blood 318 it may be smaller, or only slightly larger, in amplitude than the valve leaflet curve 318 making detection of the backflow difficult. Similar problems are encountered if the gray scale shading is used as the transition between the signal received from the tissue to the signal received from the blood, making it difficult to detect.

To enhance the presentation of a Doppler signal, processing in accordance with the invention can distinguish received power, or amplitude of the associated spectrum of the received Doppler signal. A technique that can be used to distinguish portions of the received Doppler signal based on the received power includes assigning different colors and relative brightness to different power levels. The assignment of colors or color brightness can be accomplished either automatically or manually. In addition, the assignment of color or brightness can be accomplished either directly or indirectly by a user.

In one embodiment, assignment of color or color brightness, in relation to received power level, can be accomplished through the use of a look up table. Such assignment, or separation, in terms of color with change in power may allow easier differentiation of signals received, for example differentiating signals from tissue from those from blood. Look up tables, or other techniques for assigning color or brightness based on received signal strength, can include various strategies for representation of the received signals. For example, different power levels can be mapped to different colors or color brightness levels of one or more colors.

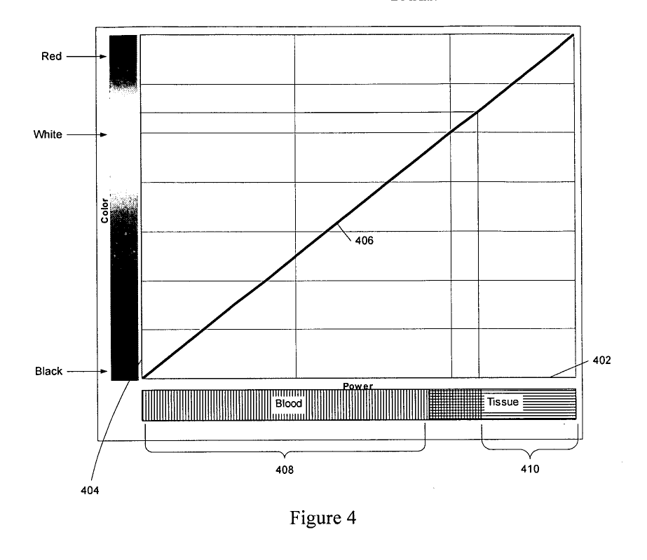

FIG. 4 is a graphical representation of a linear look-up table that maps different power levels to different colors and different brightness levels. As shown in FIG. 4, the horizontal axis 402 represents the received power level, increasing to the left. The vertical axis 404 represents varying color levels beginning with black 420 at the bottom varying to white 422 near the middle. The color then changes from white 422 to red 424, represented by stippling, at the top of the axis 402. The line 406 represents the transfer function for mapping the received power level to a desired color. For example, the strength of signals received from blood are represented by lower power levels, the right portion, 408 of the horizontal axis 402, and the strength of signals received from tissue are represented by higher power levels, the left portion, 410 of the horizontal axis 402. Using FIG. 4, signals received from blood would, in general, be mapped to black. Likewise, signals received from tissue would, in general, be mapped to red. Using different colors and color brightness levels help to differentiate signals received from blood versus signals received from tissue.

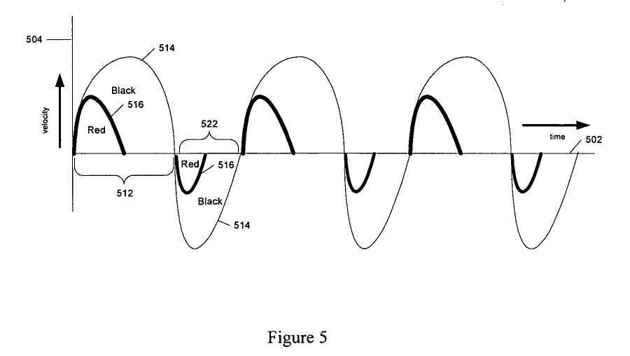

FIG. 5 is an illustration of the composite Doppler signal of FIG. 3 applying the color mapping of FIG. 4 rather than the gray scale shading of FIG. 3. FIG. 5 has a horizontal axis 502 representing time, and a vertical axis 504 representing velocity. Use of different colors, and color brightness, can improve the ability to discriminate blood flow from the heart valve leaflets. For example, as shown in FIG. 5, by mapping and displaying the signals from the blood 514 to shades of black, and signals from the valve leaflets 516 to shades of red, identified by stippling, identification of signals from the two different materials is improved. As illustrated in FIG. 5, during the period 520 when the valve leaflets are opening, the region under the curve 516 representing the valve leaflet motion to the horizontal axis 502 will be filled in shades of red indicated by stippling. During the same period the region under the curve 514 representing the blood flow to the horizontal axis 502 will be filled in shades of black to gray. Likewise, during the period 522 when the valve leaflets are closing, the region under the curve 516 will again be filled in shades of red, indicated by stippling, and the regions under the curve 514, representing regurgitation, will be filled in shades of black to gray.

As illustrated in FIG. 5, use of color can enhance the Doppler presentation. Use of color can make it easier to discriminate between Doppler signals received from different types of materials, such as blood and tissue. For example, the transition in the display between the signals representing the tissue, in red, and the signal received from the blood, in black, is easily identified.

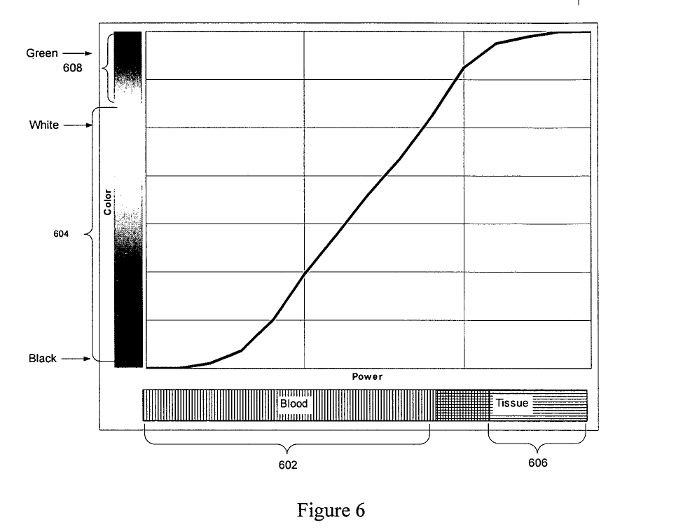

In other embodiments, non-linear mapping may also be used to improve the ability to discriminate signals from different types of materials. FIG. 6 is a graphical representation of an example of non-linear mapping of received signal strength to color. As shown in FIG. 6, lower amplitude received signal power levels, such as signals received from blood 602, are mapped to a gray scale 604 varying from black 620 to white 622 corresponding to low amplitude signals to higher amplitude signals respectively. Higher amplitude received signal power levels, such as signals received from denser material such as tissue 606, are represented in color 608. In FIG. 6, the signals received from the denser material are mapped to shades of green 608 indicated by cross hatching.

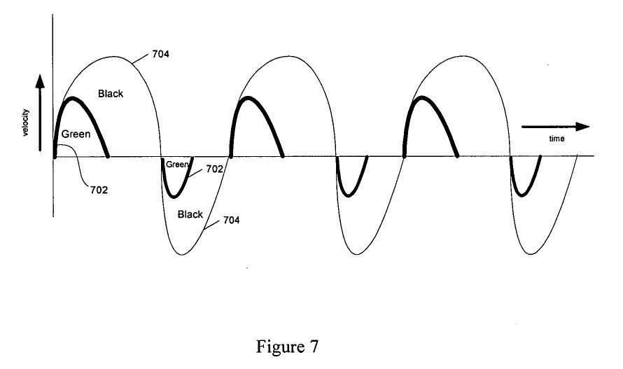

FIG. 7 is an illustration of the composite Doppler signal applying the color mapping of FIG. 6. As shown in FIG. 7, non-linear color mapping can improve the ability to discriminated between signals received from tissue 702 and signals received from blood 704. In FIG. 7, the region under the curve 702 representing signal received from tissue are represented in shades of green, indicated by cross hatching, and the region under the curve 704 representing signals received from blood are represented in shades of black to gray. Again, the use of different colors to represent Doppler signals received from different types of materials, based on received signal level, enhances the presentation of the Doppler data.

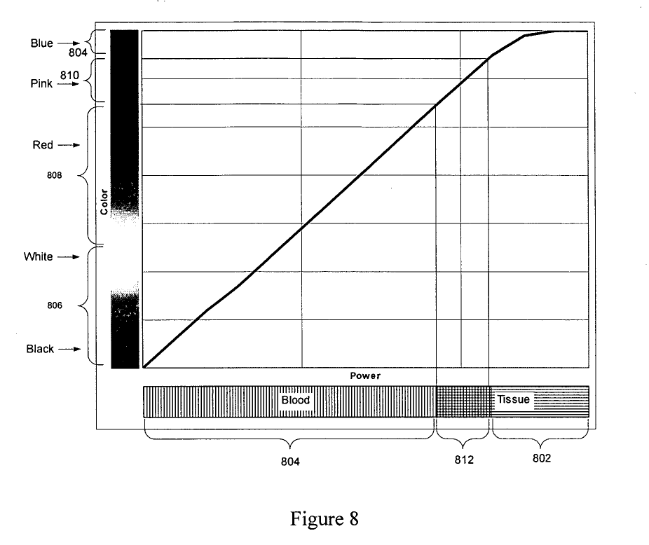

FIG. 8 is graphical representation of another example of non-linear mapping of received signal strength to a multi-colored scale. As shown in FIG. 8, higher power received signals, such as those received from tissue, 802 are selectively non-linearly compressed into a short blue scale 804, indicated by diagonal hatching. Lower power level signals, such as those received from blood, 804 are linearly spread across a gray scale 806 and a red scale 808, indicated by stippling. The higher red and red to blue transition scale 810 represent the transition from relatively lower power levels 812 from blood to the higher power levels received from tissue and are indicated by a transition from stippling to diagonal hatching. Lower level signals received from blood 804 are mapped from black to white 806 and from white to red 808, indicated by stippling.

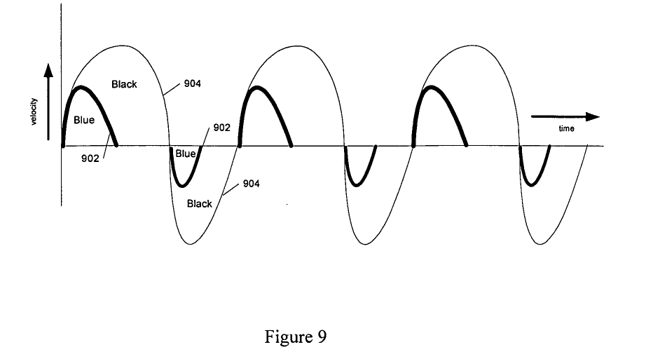

FIG. 9 is an illustration of the composite Doppler signal applying the color mapping of FIG. 8. As shown in FIG. 9, non-linear color mapping can improve the ability to discriminated between signals received from tissue 902 and signals received from blood 904. In FIG. 9, the region under the curve 902 representing signal received from tissue are represented in blue, indicated by diagonal hatching, and the region under the curve 904 representing signals received from blood are represented in shades of black to gray. Any signals received that are at power levels between those received from tissue or blood will be mapped from red to blue, indicated by overlapping of stippling and diagonal hatching. Again, the use of different colors to represent Doppler signals received from different types of materials, based on received signal level, enhances the presentation of the Doppler data.

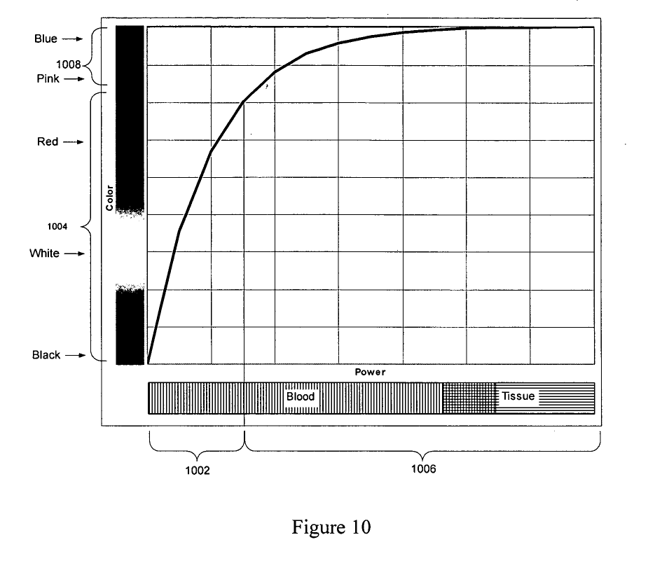

FIG. 10 is a graphical representation of yet another example of non-linear mapping of received signal strength to a multi-colored scale. As shown in FIG. 10, received signals that are at very low power levels 1002, such as those that predominantly correspond to blood, are mapped to the lower colors, approximately three-quarters of the color scale. For example, the lower level power signals can be mapped from a black to gray to red scales 1004 (red indicated by stippling). Received signals that are at higher power levels, for example those corresponding to tissue and including the transition from blood to tissue, 1006 are mapped to a blue scale 1008 indicated by diagonal hatching.

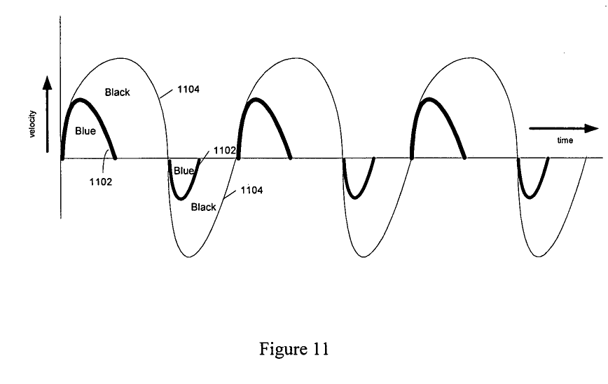

FIG. 11 is an illustration of the composite Doppler signal applying the color mapping of FIG. 10. Once again as shown in FIG. 11, non-linear color mapping can improve the ability to discriminate between signals received from tissue 1002 and signals received from blood 1004. In FIG. 11, the region under the curve 1102 representing signal received from tissue are represented in blue, indicated by diagonal hatching, and the region under the curve 1104 representing signals received from blood are represented in shades of black to gray to red, where red is indicated by stippling. Comparison of FIGS. 9 and 11 show that the representation of signals received from blood in FIG. 11 have more resolution because of the non-linear mapping shown in FIG. 10 where an expanded portion of the color bar is used to represent the signal received from blood. By expanding the color map used to represent the signals received from blood additional details about the blood flow may be observed. A similar technique where the color map used to represent the signals received from tissue could be used such that additional details about the tissue movement may be observed. Again, the use of different colors to represent Doppler signals received from different types of materials, based on received signal level, enhances the presentation of the Doppler data.

It is noted that the use of linear or non-linear mapping, or compression, may be desired depending on the overall system configuration. For example, a linear compression algorithm, as illustrated in FIG. 4, may be desired where either a linear or a nonlinear signal is mapped to color. If non-linear signal compression is already applied, say as part of the Doppler processing or as part of earlier processing, a linear look up table as shown in FIG. 4 may be more suitable than a non-linear mapping. Likewise, if there has been no compression previously applied to the signal, then a non-linear compression may be desired. Also, non-linear compression may be desired even if there has been previous processing of the signal.

The techniques described can be implemented as part of any system that allows processing of Doppler data to distinguish the power, or amplitude of the spectrum, of a Doppler signal and then assign color, or relative color brightness, for presentation of the Doppler data. The presentation may be either directly or indirectly presented to a user. For example, a system can utilize Doppler processing capabilities of an host ultrasound scanner to obtain a time-varying signal representative of the velocity of flow, for example blood flow, through an area of interest. Such areas of interest can include, especially in the case of imaging the heart, valves and other moving tissue structures and blood. Mapping the received signal strength to different colors and color brightness, for example by using a look-up table, makes it easier differentiation of signals from tissue to those from blood.

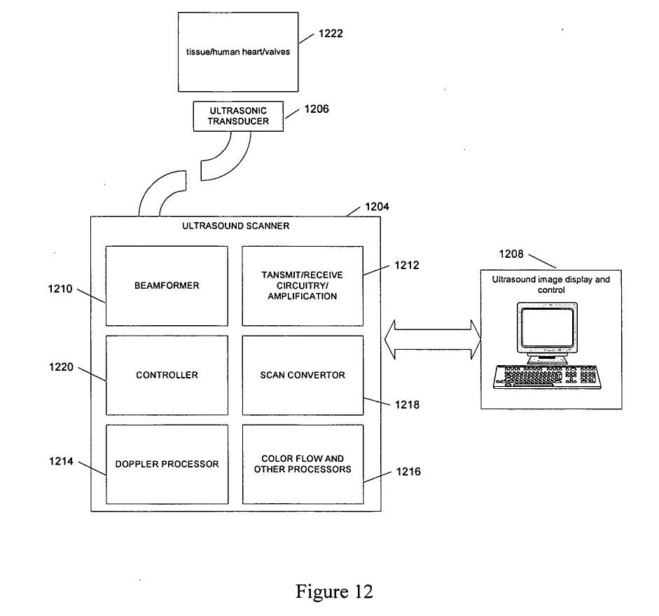

The techniques described can be implemented in many different systems. FIG. 12 is a block diagram illustrating an embodiment of a Doppler scanner system 1202 constructed in accordance with the present invention. The system 1202 includes an ultrasound scanner 1204, an ultrasonic transducer 1206, and a display 1208. The ultrasound scanner 1204 can be capable of intercepting and interpreting Doppler signals. The ultrasound scanner 1204 may include various circuits and subsystems for performing various functions. For example, the ultrasound scanner 1206 can include beam former 1210 and tranmit/receivce 1212 circuits or subsystems. The ultrasound scanner 1204 may also include a Doppler processor 1214, and color flow and other processor 1216. The ultrasound scanner 1204 may also include a scan converter 1218 and a control 1220.

The ultrasound scanner 1204 generates signals that are communicated to the ultrasonic transducer 1206. The ultrasonic transducer transmits and receives signals from a desired sample 1222, for example from a human heart tissue and blood. Signals received by the ultrasonic transducer 1206 are communicated to the ultrasound scanner 1204. In one embodiment, the ultrasound scanner 1204 processes the received signals, including color mapping, and the processed signal is communicated to the display 1208 for presentation to a user. In another embodiment, the ultrasound scanner 1204 does some processing of the received signal and the display 1208 includes a processor that does some processing of the signal, for example color mapping, before presentation to a user. In general, the ultrasound scanner 1204 includes a combination of digital or analog electronics capable of generating necessary signals and processing such received signals so as to generate Doppler representations in accordance with the invention. In addition, processing of the Doppler signals may be performed real-time, that is at the time the signals are captured, or off-line following the capture of the data.

The ultrasonic transducer 1206 can include, for example, one or more transducers that utilizes piezoelectric properties to generate acoustic signals from electrical signals. The transducer may be a mechanical, sector, linear, or curved array designs. In general, the type of transducer used is selected to be appropriate for the particular application such as external application, trans-oesophageal, intra-vascular, intra-cardiac, or endocavitary applications.

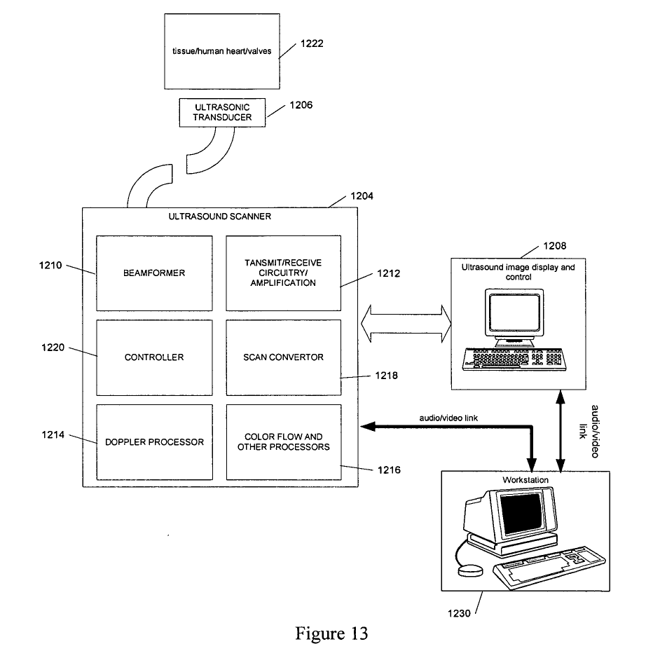

FIG. 13 is a block diagram of the Doppler scanner system of FIG. 12 and includes a workstation 1230. In the embodiment of FIG. 13, the workstation 1230 may include hardware and/or software that exists separate from the ultrasound scanner 1204. The workstation 1230 may be in communication with the ultrasound scanner 1204, the display 1208, or both. For example, video, audio, or both may be communicated between the ultrasound scanner 1204 and the display 1208. Communication between the workstation 1230, the display 1206 and the ultrasound scanner 1204 can include video, audio, Electrocardiogram (ECG) signals, or other types of signals in either digital and/or analog format. The above described techniques can then be performed either partially or entirely on the workstation 1230

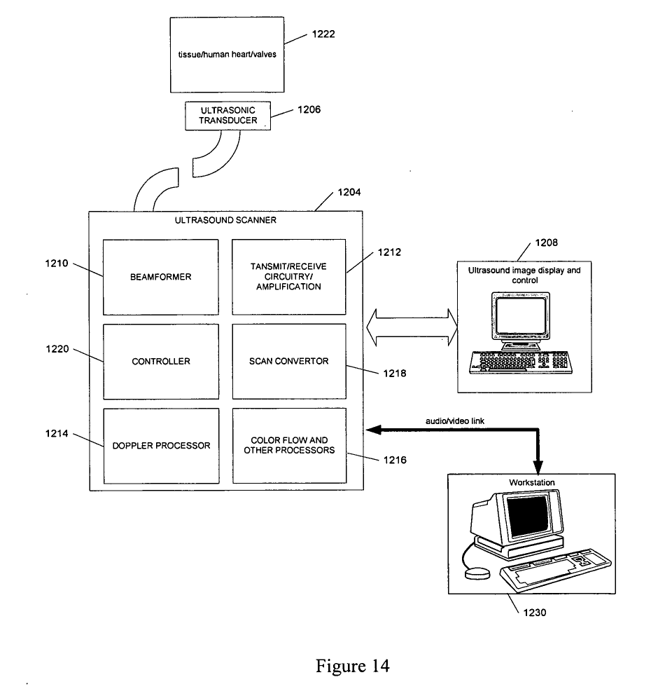

FIG. 14 is a block diagram of another embodiment of a Doppler scanner system. In the embodiment illustrated in FIG. 14 the workstation 1230 communicates only with the ultrasound scanner 1204.

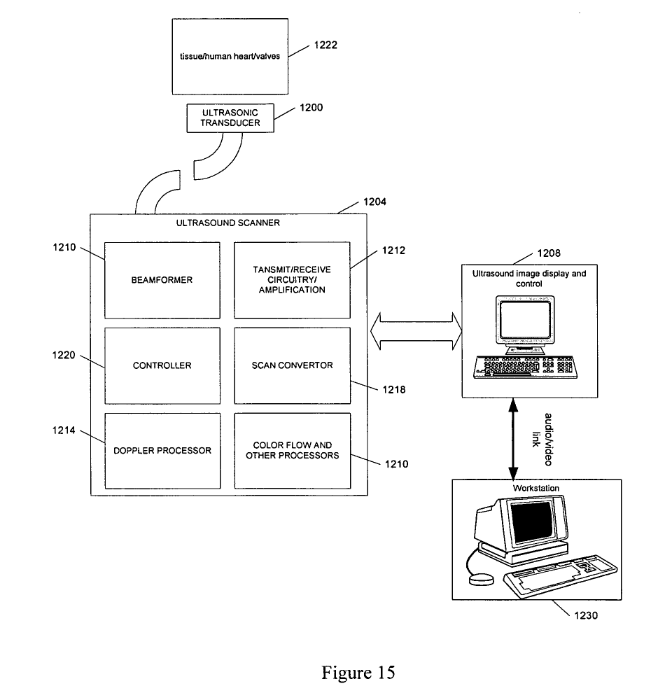

FIG. 15 is a block diagram of yet another embodiment of a Doppler scanner system. In the embodiment illustrated in FIG. 14, the workstation 1230 communicates only with the display 1208.

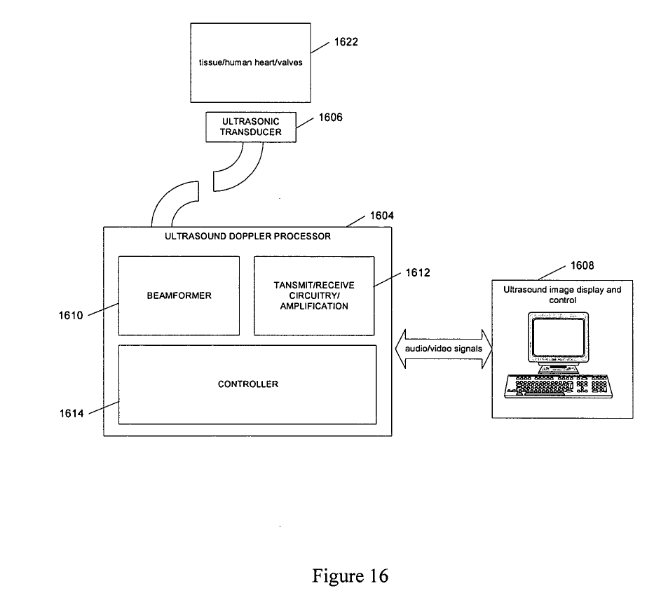

The previous embodiments describe a general Doppler scanner system. A system could also be implemented using a simple ultrasound Doppler processing set up. FIG. 16 is a block diagram illustrating another embodiment of a Doppler scanner system 1602 constructed in accordance with the present invention. The system 1602 includes an ultrasound Doppler processor 1604, an ultrasonic transducer 1606, and a display and control 1608. The ultrasound Doppler processor 1604 can be capable of intercepting and interpreting Doppler signals. The ultrasound Doppler processor 1604 may include various circuits and subsystems for performing various functions. For example, the ultrasound Doppler processor 1606 can include beam former 1610, tranmit/receivce 1612 circuits or subsystems, and a controller 1614. The ultrasound Doppler processor 1604 may generate signals that are communicated to the ultrasonic transducer 1606. The ultrasonic transducer transmits and receives signals from a desired sample 1622, for example from a human heart tissue and blood. Signals received by the ultrasonic transducer 1606 are communicated to the ultrasound Doppler processor 1604. In one embodiment, the ultrasound Doppler processor 1204 processes the received signals, including color mapping, and the processed signal is communicated to the display 1208 for presentation to a user. In another embodiment, the ultrasound Doppler processor 1604 does some processing of the received signal and the display 1608 includes a process that does some processing of the signal, for example color mapping, before presentation to a user.

Other combinations of hardware and software may be used to perform the techniques described so as to achieve the operationality described. For example, there are multiple ways of interlinking the components that form this invention.

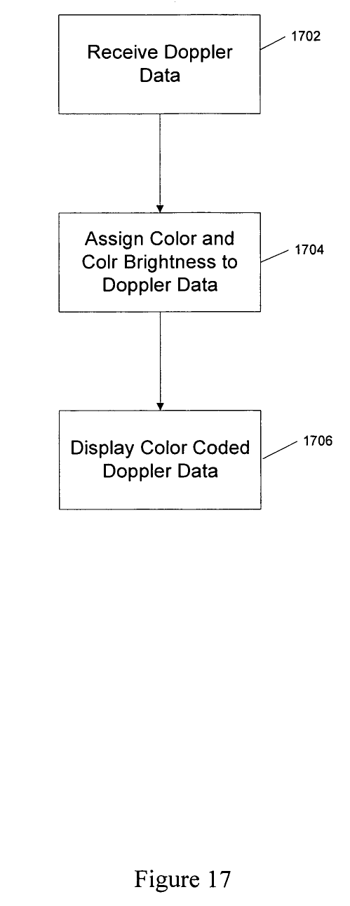

FIG. 17 is a flow diagram illustrating a method of enhancing the presentation of Doppler signals. Flow begins in block 1702 where data from Doppler signals is received. Flow then continues to block 1704 where color, and color brightness are assigned to the Doppler data in accordance with the strength of the received Doppler signal. In one embodiment, a look-up table is used to map the Doppler signal strength to a particular color and color brightness. In other embodiments other techniques are used to assign color to the Doppler signal in accordance with the strength of the Doppler signal. In one embodiment the assignment of color and color brightness are linear in relationship to the signal strength. In other embodiments the assignment of color and color brightness is non-linear in accordance with the strength of the Doppler signal. Flow then continues to block 1706 where the color Doppler data is displayed.

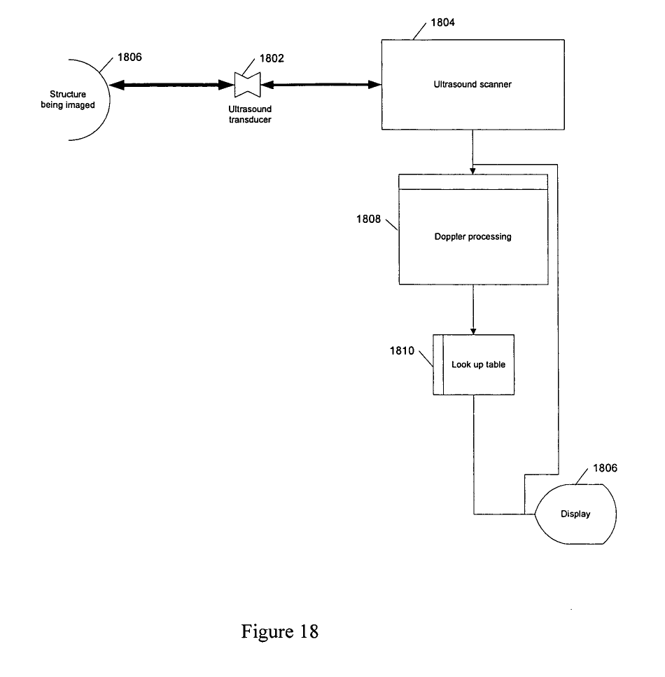

FIG. 18 is a block diagram illustrating a method of processing Doppler signals. As shown in FIG. 18, an ultrasound transducer 1802 is in communication with a an ultrasound scanner 1804. The ultrasound transducer is also in communication with a structure 1804 being imaged. Commands from the ultrasound scanner 1804 are communicated to the ultrasound transducer which transmits and receives ultrasound signals to the structure 1806 being imaged. The signals received by the ultrasound transducer 1802 are communicated to the ultrasound scanner 1804. The ultrasound scanner 1804 may do some processing of the signals received from the ultrasound transducer 1802. For example the ultrasound scanner 1804 may generate a B-mode or an M-mode display and route it to a display 1806. The ultrasound scanner 1804 may pass the signal received from the ultrasound transducer 1802 directly to a Doppler processor 1808 or the ultrasound scanner 1804 may do some processing of the signals before sending them to the Doppler processor 1808.

The Doppler processor 1808 processes the signals received from the ultrasound scanner 1804. For example, the Doppler processor 1808 may discriminate the signals based upon the amplitude of the received signal strength. The Doppler processor may use a look-up table 1810 to map the Doppler signals to different colors based upon the received signal strength. The look-up table may be either linear or non-linear. The color mapped Doppler data is then sent to the display 1806 for presentation.

Those of skill in the art will understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill in the art will further appreciate that the various illustrative modules, circuits, and algorithms described may be implemented as electronic hardware, computer software, or combinations of both. Also, the various modules and circuits described may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, any conventional processor, controller, or micro-controller. A processor may also be implemented as a combination of computing devices, e.g., a combination of a DSP and a microprocessor, a plurality of microprocessors, one or more microprocessors in conjunction with a DSP core, or any other such configuration. Software modules may reside in RAM memory, flash memory, ROM memory, EPROM memory, EEPROM memory, registers, hard disk, a removable disk, a CD-ROM, or any other form of storage medium known in the art.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A method of processing Doppler signals, the method comprising:
determining signal strength of received Doppler signals; and
assigning color to the received Doppler signals in accordance with the strength of the received Doppler signal.

2. A method as defined in claim 1, wherein Doppler data that has been assigned color is presented to a user.

3. A method as defined in claim 1, wherein a look-up table is used to assign color.

4. A method as defined in claim 1, wherein assigning color from a range of color values is linear in relation to the strength of the received Doppler signal.

5. A method as defined in claim 1, wherein assigning color from a range of color values is non-linear in relation to the strength of the received Doppler signal.

6. A method as defined in claim 1, wherein assigning color further comprises assigning color brightness.

7. A method as defined in claim 1, wherein the received Doppler signal includes signals reflected from different types of materials.

8. A method as defined in claim 1, wherein the received Doppler signals include signals reflected from tissue.

9. A method as defined in claim 1, wherein the received Doppler signals include signals reflected from a heart valve.

10. A method as defined in claim 1, wherein the received Doppler signals include signals reflected from blood.

11. A method as defined in claim 1, wherein the Doppler signals are received from an ultrasound transducer that is included in a catheter.

12. A method of processing Doppler ultrasound signals, the method comprising:
receiving Doppler ultrasound data that includes strength of a received Doppler ultrasound signal;
assigning color to the received Doppler ultrasound data in accordance with the strength of the received Doppler ultrasound signal such that different ranges of received signal strengths are assigned different colors.

13. A method as defined in claim 12, wherein Doppler data that has been assigned color is presented to a user.

14. A method as defined in claim 12, wherein a look-up table is used to assign color.

15. A method as defined in claim 12, wherein the different ranges of received signal strength correspond to different types of material the Doppler ultrasound signal is reflected from.

16. A method as defined in claim 15, wherein a type of material is tissue.

17. A method as defined in claim 15, wherein a type of material is blood.

18. A method as defined in claim 12, wherein the Doppler data is received from an ultrasound transducer that is included in a catheter.

19. An ultrasound system comprising:
an ultrasonic transducer configured to transmit and receive ultrasonic signals from a sample;
an ultrasound scanner configured to communicate signals to the ultrasonic transducer to be transmitted into the sample, wherein the scanner receives signals from the ultrasonic transducer that were reflected from objects within the sample, the scanner processes the received signals to determine velocity of an object reflecting signals based on Doppler effect and assign color to represent the received signal based on the strength of the received signal; and
a display for displaying the Doppler data that has been assigned color.

20. An ultrasound system as defined in claim 19, wherein the sample is a biological tissue.

21. An ultrasound system as defined in claim 19, further comprising multiple objects reflecting signals.

22. An ultrasound systems as defined in claim 19, wherein the received signals are reflected from different types of materials.

23. An ultrasound system as defined in claim 22, wherein one of the materials is tissue.

24. An ultrasound system as defined in claim 22, wherein one of the materials is blood.

25. An ultrasound system as defined in claim 19, wherein the ultrasound transducer is included in a catheter.

26. An ultrasound system comprising:
an ultrasonic transducer configured to transmit and receive ultrasonic signals from a sample;
an ultrasound scanner configured to communicate signals to the ultrasonic transducer to be transmitted into the sample, the scanner receives signals from the ultrasonic transducer that were reflected from objects of the sample;
a processor configured to processes the received signals to determine a velocity of an object reflecting signals based on Doppler effect and assigning color to represent the received signal based on the strength of the received signal; and
a display for displaying Doppler data that has been assigned color.

27. An ultrasound system as defined in claim 26, wherein the processor is in a workstation.

28. An ultrasound system as defined in claim 26, wherein the processor is in the ultrasound scanner.

29. An ultrasound system as defined in claim 26, wherein the processor and display are in a workstation.

30. An ultrasound system as defined in claim 26, wherein the ultrasound transducer is included in a catheter.

31. A method of representing Doppler signals, the method comprising:
means for determining signal strength of received Doppler signals; and
means for assigning color to the received Doppler signals in accordance with a strength of the received Doppler signal.

32. A method of processing Doppler ultrasound signals, the method comprising:
means for receiving Doppler ultrasound data that includes a strength of a received Doppler ultrasound signal;
means for assigning color to the received Doppler ultrasound data in accordance with the strength of the received Doppler ultrasound signal such that different ranges of received signal strengths are assigned different colors.

33. An ultrasound system comprising:
a catheter mounted ultrasonic transducer configured to transmit and receive ultrasonic signals from a sample;
an ultrasound scanner configured to communicate signals to the ultrasonic transducer to be transmitted into the sample, the scanner receives signals from the ultrasonic transducer that were reflected from objects within the sample;
a processor configured to processes the received signals to determine a velocity of an object reflecting signals based on Doppler effect and assigning color to represent the received signal based on the strength of the received signal; and
a display for displaying Doppler data that has been assigned color.

34. A computer readable media embodying a method of encoding data, the method comprising:
determining signal strength of received Doppler signals; and
assigning color to the received Doppler signals in accordance with a strength of the received Doppler signal.
